(19) United States
(12) Patent Application Publication
(10) Pub. No.: US 2021/0100380 A1
(43) Pub. Date: Apr. 8, 2021

(54) HYGIENIC TRAVEL HEADREST ACCESSORY SYSTEM

(71) Applicant: Location A.L.L. incorporée, Gatineau (CA)

(72) Inventor: Anne-Lise Massie-Plante, Gatineau (Quebec) (CA)

(73) Assignee: Location A.L.L. incorporée, Gatineau (CA)

(21) Appl. No.: 17/125,718

(22) Filed: Dec. 17, 2020

Related U.S. Application Data
(63) Continuation of application No. PCT/CA2019/050858, filed on Jun. 19, 2019.
(60) Provisional application No. 62/811,483, filed on Feb. 27, 2019.

(30) Foreign Application Priority Data
Jun. 20, 2018 (CA) 3008966

(51) Int. Cl. A47G 9/02; A47G 9/10; A61F 9/04

(52) U.S. Cl. CPC A47G 9/0253; A61F 9/04; A47G 9/1045

(57) ABSTRACT

Disclosed herein is a hygienic travel headrest accessory system that can be used when travelling by plane, car, bus, train, etc. to enable a traveller to adapt any travel seat to hygienically protect their head while providing a convenient system for organizing travel accessories. The traveller can use the cinching mechanism to both configure a headrest strap to cinch around the headrest so as to ensure that the headrest strap remains in place when cinched around the headrest while positioning the textile component hygienically between the headrest and the travellers head and to secure the accessory attachment component for receiving travel accessories in a “stowed and at ready” position or in an “in use” position.

### TECHNICAL FIELD

This application relates to textile travel accessories in general, and to a hygienic travel headrest accessory system, in particular.

### SUMMARY

According to one aspect of the present disclosure, there is provided a hygienic travel headrest accessory system that can be used when travelling by plane, car, bus, train, etc. to enable a traveller to adapt any travel seat to hygienically protect their head while providing a convenient system for organizing travel accessories. More particularly, there is provided a hygienic travel headrest accessory system including an elongate textile component having an operative side for covering a portion of a headrest of the seat of a traveller and a manipulative side for abutting against the head of the traveller. The system further includes a headrest strap connected to the textile component at one end and extending freely for a predetermined distance thereafter sufficient to go around the headrest, the headrest strap having an operative side with a non-slip component; a cinching mechanism connecting the free end of the headrest strap to the other end of the textile component for cinching the headrest strap and textile component around the headrest thereby positioning the textile component between the headrest and the head of the traveller; and an accessory attachment component provided at least on one end of the textile component for receiving travel accessories. The traveller can use the cinching mechanism to both configure the headrest strap to cinch around the headrest so as to ensure that the headrest strap remains in place when cinched around the headrest while positioning the textile component hygienically between the headrest and the travellers head and to secure the accessory attachment component for receiving travel accessories in a “stowed and at ready” position or in an “in use” position.

Other aspects and features of the present disclosure will become apparent to those ordinarily skilled in the art upon review of the following description of specific embodiments of a hygienic travel headrest accessory system in conjunction with the accompanying drawing figures.

### BRIEF DESCRIPTION OF THE DRAWINGS

Embodiments of the present disclosure will now be described, by way of example only, with reference to the accompanying drawing figures, wherein:

FIG. 1 is a front left perspective view of an example pair of airline seats;

FIG. 2 is a front perspective view of a seat of FIG. 1;

FIG. 3 is a front left perspective view of a headrest of the seat of FIG. 2;

FIG. 4 is a right rear perspective view of a headrest of the seat of FIG. 2;

FIG. 5 is a top perspective view of a seatrest of seat of FIG. 2;

FIG. 6 is a top perspective view of an operational side of an example hygienic headrest accessory system;

FIG. 7 is a top perspective view of a manipulative side of the example hygienic headrest accessory system of FIG. 6;

FIG. 8 is a front perspective view of the example hygienic headrest accessory system of FIG. 6 operating on the seat of FIG. 2;

FIG. 9 is a front left perspective view of the example hygienic headrest accessory system of FIG. 6 operating on the seat of FIG. 2;

FIG. 10 is a right rear perspective view of the example hygienic headrest accessory system of FIG. 6 operating on the seat of FIG. 2;

FIG. 11 is a front perspective view of an example accessory attachment component;

FIG. 12 is a front perspective view of the example accessory attachment component of FIG. 11 with a strap attached;

FIG. 13 is a front perspective view of an example eye mask accessory component using two of the accessory attachment components of FIG. 11;

FIG. 14 is a front perspective view of the example hygienic headrest accessory system of FIG. 6 operating on the seat of FIG. 2 and the eye mask accessory of FIG. 13;

FIG. 15 is a front perspective view of an example accessory attachment component;

FIG. 16 is a front perspective view of a three example accessory attachment components of FIG. 15 for improved manipulation of three different example travel accessories;

FIG. 17 is a front perspective view of the example multiple accessory attachment component of FIG. 15 for improved manipulation of three different example travel accessories;

FIG. 18 is a front perspective view of the example hygienic headrest accessory system of FIG. 6 co-operating with the seat of FIG. 2, the eye mask accessory of FIG. 13, and the multiple accessory attachment component of FIG. 15;

FIG. 19 is a front perspective view of an alternative example hygienic headrest accessory system co-operating with the seat of FIG. 2, the eye mask accessory of FIG. 13, and integrating the multiple accessory attachment feature of FIG. 15;

FIG. 20 is a front perspective view of an alternative example of FIG. 12 integrating the multiple accessory attachment feature of FIG. 15;

FIG. 21 is a top perspective view of an operational side of an alternative example hygienic headrest accessory system;

FIG. 22 is a top perspective view of a pocket feature of the alternative example hygienic headrest accessory system of FIG. 21;

FIG. 23 is a top perspective view of a reversible storage feature of the alternative example hygienic headrest accessory system of FIG. 21;

FIG. 24 is a front perspective view of an example accessory attachment component having a D-ring;

FIG. 25 is a front perspective view of an example eye mask accessory component using two of the accessory attachment components of FIG. 24 to attach to an example hygienic headrest accessory system operating on the seat of FIG. 2;

FIG. 26 is a right rear perspective view of the example hygienic headrest accessory system of FIG. 25 operating on the seat of FIG. 2;

FIG. 27 is a front left perspective view of the example hygienic headrest accessory system of FIG. 25 operating on the seat of FIG. 2;

FIG. 28 is a front perspective view of the example eye mask accessory component of FIG. 25 using two of the accessory attachment components of FIG. 24;

FIG. 29 is a front perspective view of an alternative to the example hygienic headrest accessory system of FIG. 25; and

FIG. 30 is a top perspective view of a pocket feature of the example hygienic headrest accessory system of FIG. 29.

Like reference numerals are used in different figures to denote similar elements.

### DETAILED DESCRIPTION OF THE DRAWINGS

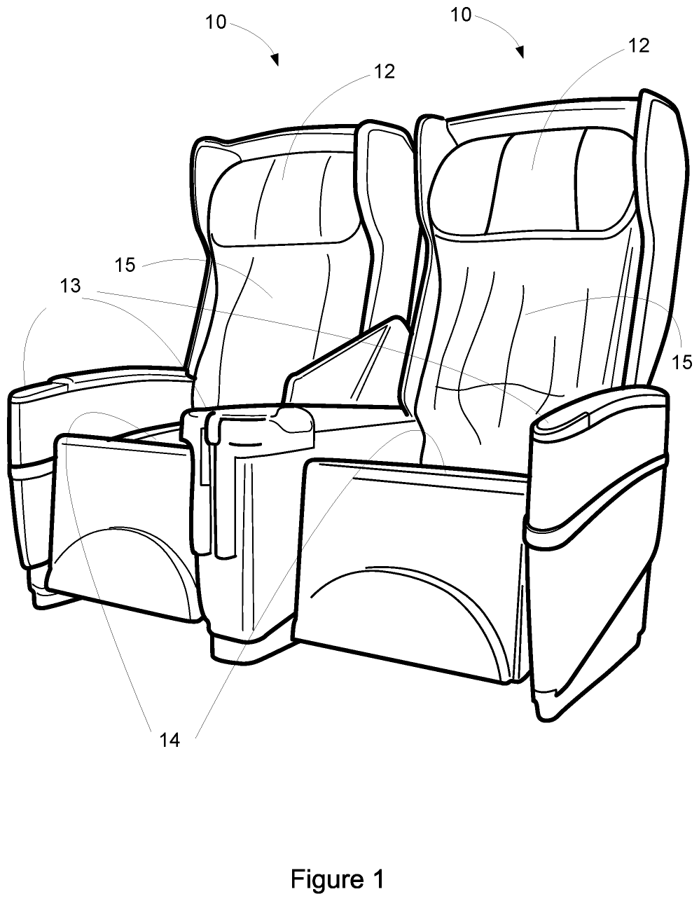

Referring to the drawings, FIG. 1 is a front left perspective view of an example pair of airline seats. As illustrated, the seats include a headrest 12, a backrest 15, a seatrest 14, and one or more armrest 13. A typical example of this kind of seating is found on commercial aircraft, busses, trains, automobiles, etc. Typically, there are jacks for earphones provided in the armrests to be used to listen to music, or more generally audio such as from an on-board entertainment system, while seated, usually when the seatbelt light is turned off. Although not shown, the back of the backrest 15 of the seat 10 immediately in front of a given seat 10 may include an on-board entertainment system that the traveller can control.

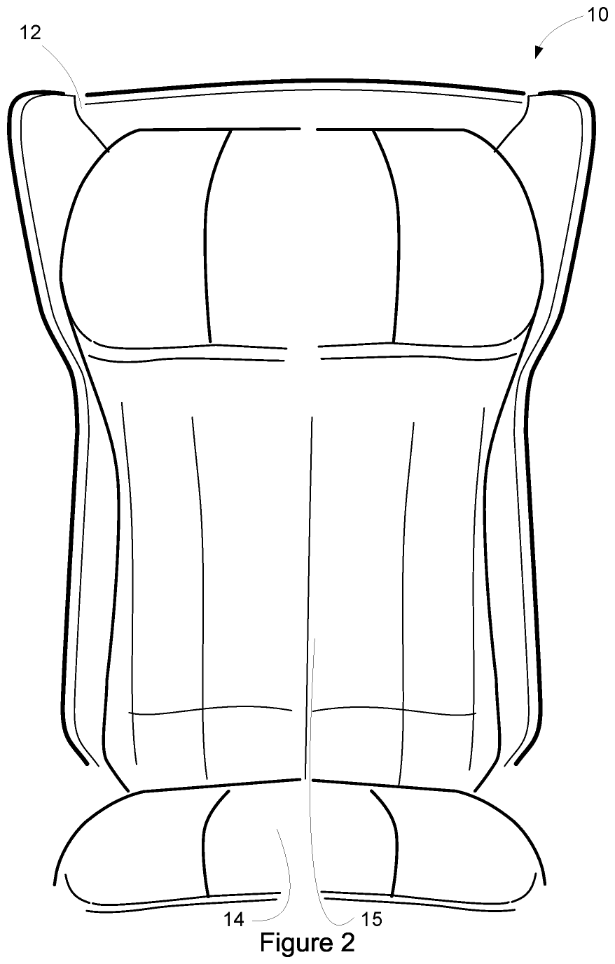

FIG. 2 is a front perspective view of one of the seats of FIG. 1. As illustrated the seat 10 includes a headrest 12 where a traveller rests their head, a backrest 15, and a seatrest 14. One difficulty a traveller faces when travelling is that space is at a premium in their seat 10, and it can be difficult to organize all the accessories one travels with such as an eye mask, earphones, blanket 38, headphones 36 etc. Another difficulty a traveller faces when travelling is that they must rest their head on a headrest 12 that has been extensively used by other travellers. Being proximate to eyes, mouth, nose, hair, and ears, the headrest 12 is very unhygienic. Ye another difficulty a traveller faces when travelling is that they may fall asleep and if they are in a bad posture their sleep can be very uncomfortable, non-ergonomic, and even cause a kink or pain in their neck and back due to sleeping in an awkward position. What's more, a sleeping traveller may inadvertently drift into the seat 10 space of a neighbouring further causing discomfort to a typical traveller.

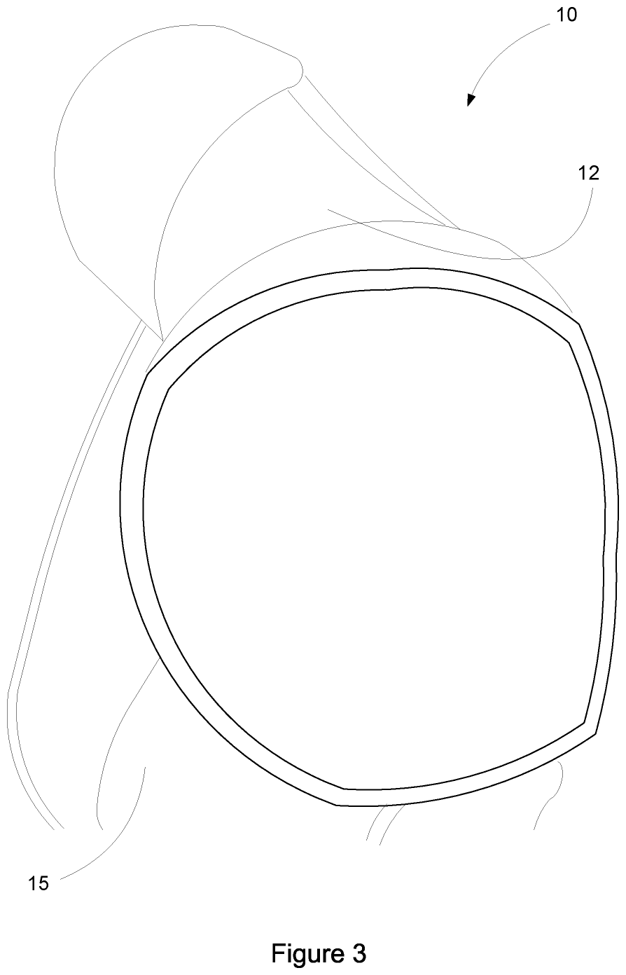

FIG. 3 is a front left perspective view of the headrest 12 of the seat 10 of FIG. 2. As illustrated, the headrest 12 has a substantially concave shape to receive the head of a traveller, being raised on the edges, and shallower in the centre. Although this is the shape illustrated, the present disclosure works with all manner of headrest 12, be they concave as illustrated, concave, integrated, fixed, or attached to the seat 10, so long as the top of the headrest 12 is positioned within a predetermined distance, e.g. 4 inches, from the top of the head of a traveller. In some seats, there is no clear distinction between the backrest 15 and the headrest 12, such that what is meant as a headrest 12 in those kinds of seat 10, is the top of the backrest 15.

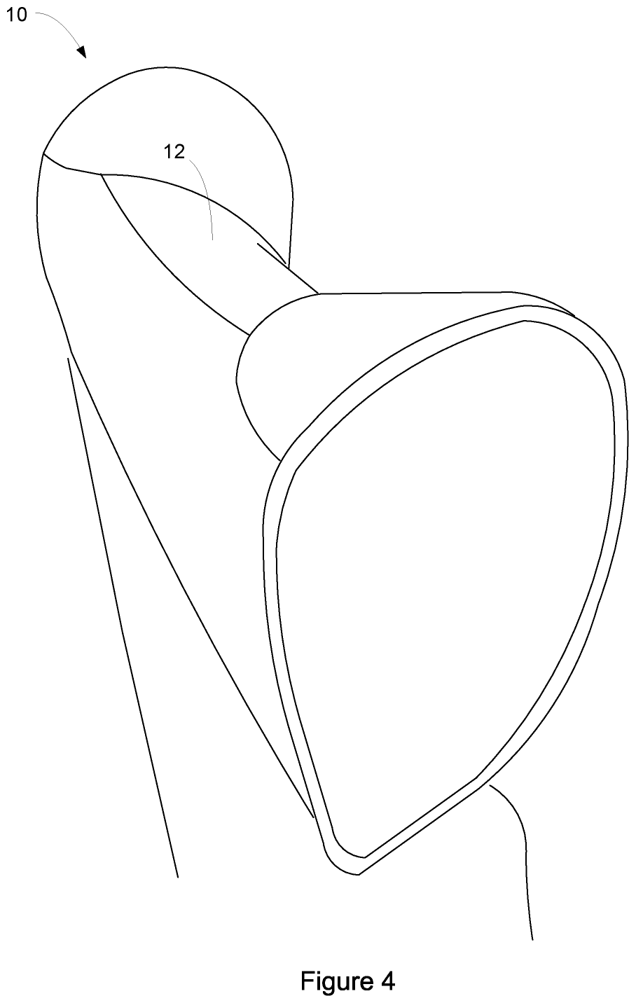

FIG. 4 is a right rear perspective view of the headrest 12 of the seat 10 of FIG. 2. The back of headrest 12 is typically flat or concave, but even in the case of a convex back of a headrest 12, the techniques of the present disclosure are effective. Although not shown in the drawing, in some seats, such as in particular airline seats, and in some vehicle, e.g. car seats, an on-board entertainment system may be provided. Advantageously, the techniques of the present invention work with such headrest.

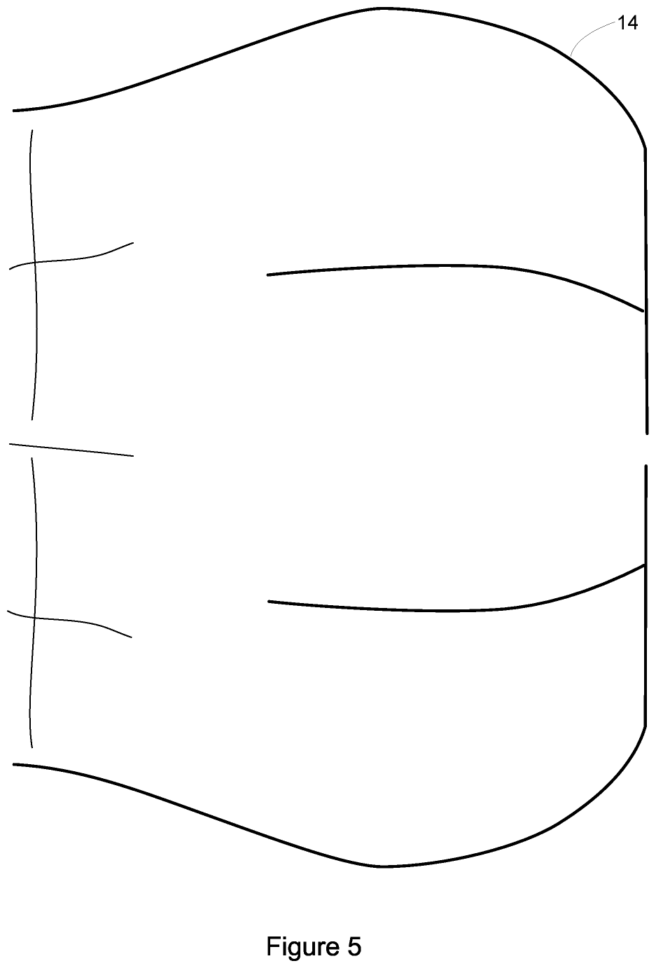

FIG. 5 is a top perspective view of the seatrest 14 of the seat 10 of FIG. 2. As illustrated, the footprint that a traveller has when seen in two dimensions is quite limited.

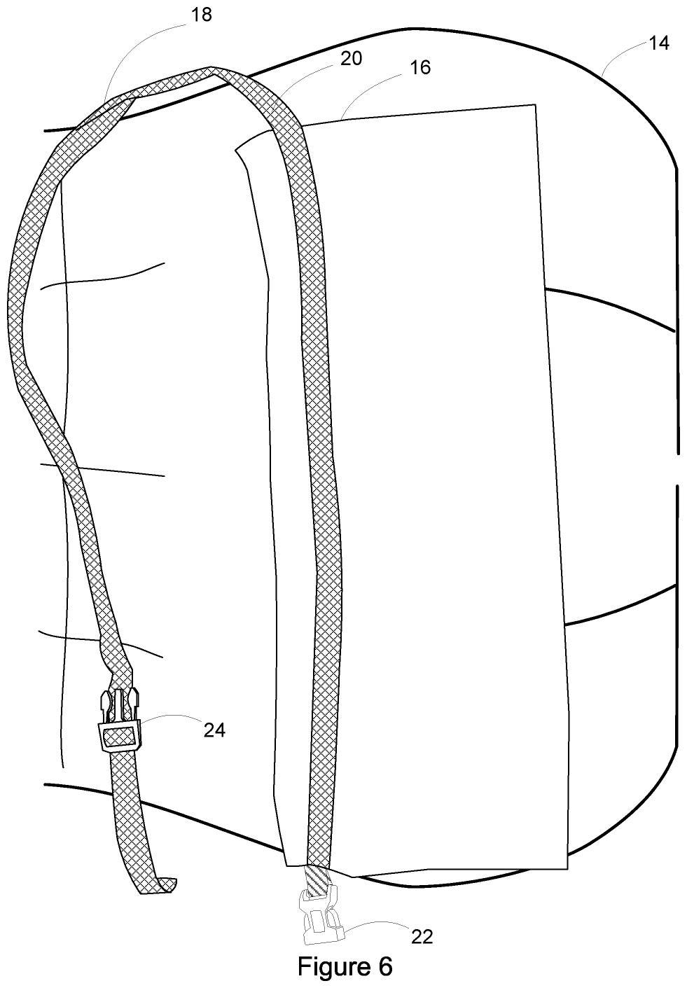

FIG. 6 is a top perspective view of the operational side of an example hygienic headrest 12 accessory system. As illustrated, the system is illustrated on the seatrest 14 to provide a sense of scale in the limited footprint available to a traveller. As illustrated, a main elongate rectangular textile component 16 is illustrated and, in one example embodiment, measures 6 inches high by 10 inches wide. Other sizes are considered to be within the scope of the present application. Onto this rectangular textile component 16 is sewn a headrest strap 18, which as illustrated, is sewn throughout its full length a small distance from the top of the rectangular textile component 16, and extending freely for a predetermined distance thereafter. In alternative embodiments, the headrest strap 18 need not be sewn on the full length of the rectangular textile component 16 and may be provided in two parts, each part sewn only on respective ends of the rectangular textile component 16. In alternative embodiments, the headrest strap 18 could be elastic, or be made of a non-elastic material. In the example embodiment, the headrest strap 18 measures ½ inch in height, is at 1 inch from the top, and is of a predetermined length sufficient to go around the headrest 12, e.g. 24 inches in length. In some embodiments, the headrest strap 18 includes an elastic component, such as is used in waistbands in the textile industry. As illustrated, the side that is exposed of the headrest strap 18 and that is meant to operate on the headrest 12 includes a non-slip component 20, such as a rubber coating, to ensure that the headrest strap 18 remains in place when cinched around the headrest 12. At one side of the rectangular textile there is provided a releasable buckle 22 component that is compatible with a corresponding releasable buckle 24 component provided on the loose end of the headrest strap 18 that extends beyond the end of the rectangular textile component 16. The rectangular textile component 16 is made of a material that is comfortable for a traveller's head to rest against, such as for example polar fleece or cotton. The choice of materials can be adapted to the materials of a specific seat 10, such as if the seat 10 is made of fabric or leather, etc. Advantageously, the rectangular textile component 16 provides a hygienic covering to the headrest 12 for the traveller since the side that abuts against the headrest 12, the operative side, is always different than the side that abuts against the traveller's head, the manipulative side, as will be more clearly shown in other figures.

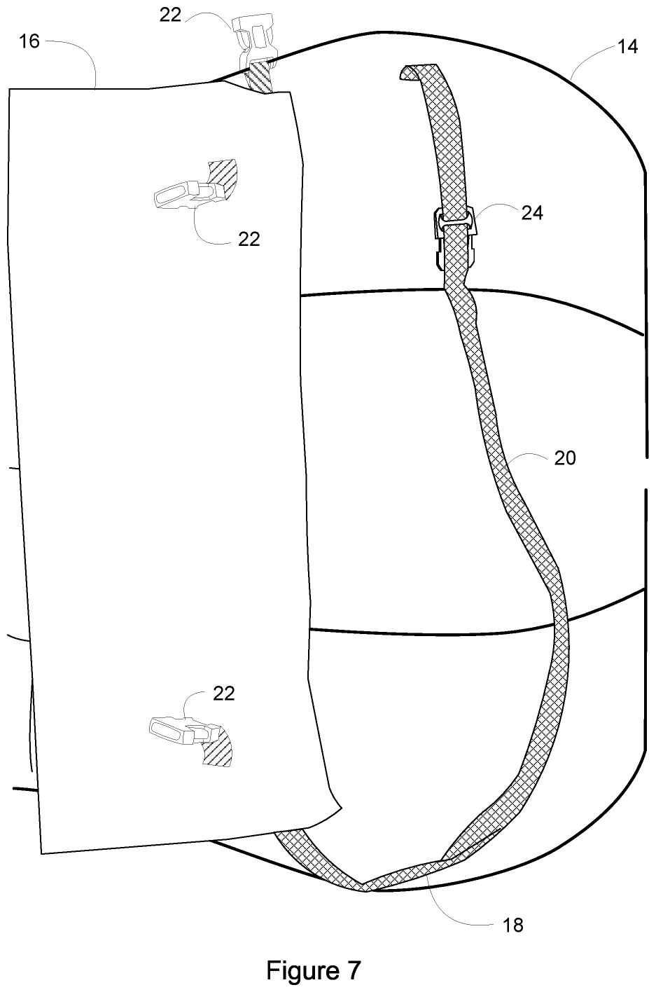

FIG. 7 is a top perspective view of the manipulative side of the example hygienic headrest 12 accessory system of FIG. 6. As illustrated, the other side of the rectangular textile component 16 includes a pair of releasable buckle 22 components provided near opposite ends of the rectangular textile component 16, a fixed distance from each side and the top of the rectangular textile component 16. For example, as illustrated the releasable buckles are provided an inch from each side and the top of the rectangular textile component 16. Advantageously, the releasable buckles provide a manipulative solution for manipulating travel accessories, as will be illustrated further in the following figures.

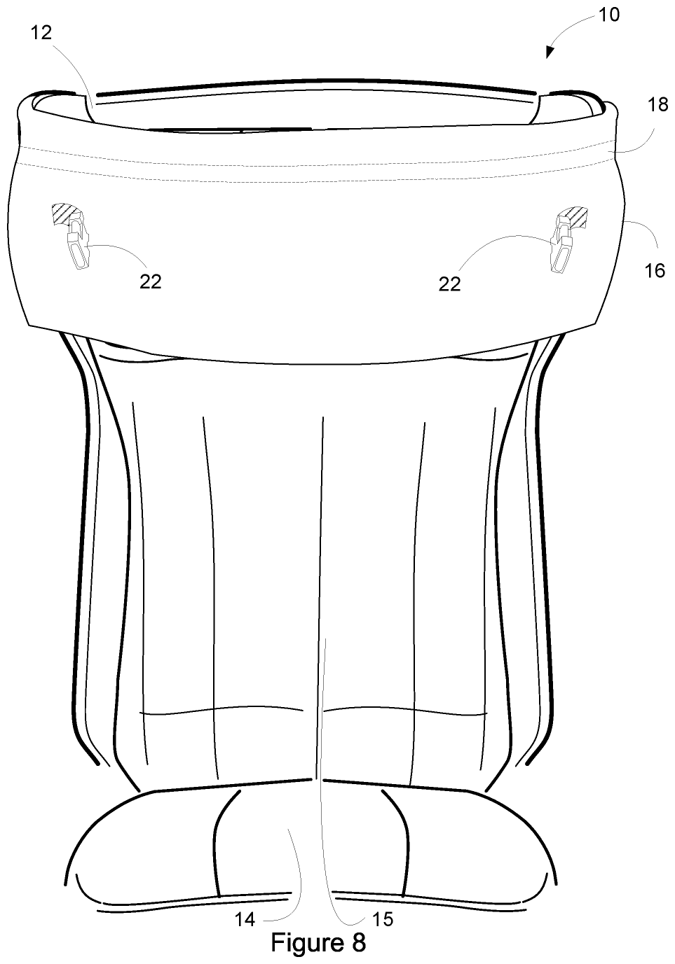

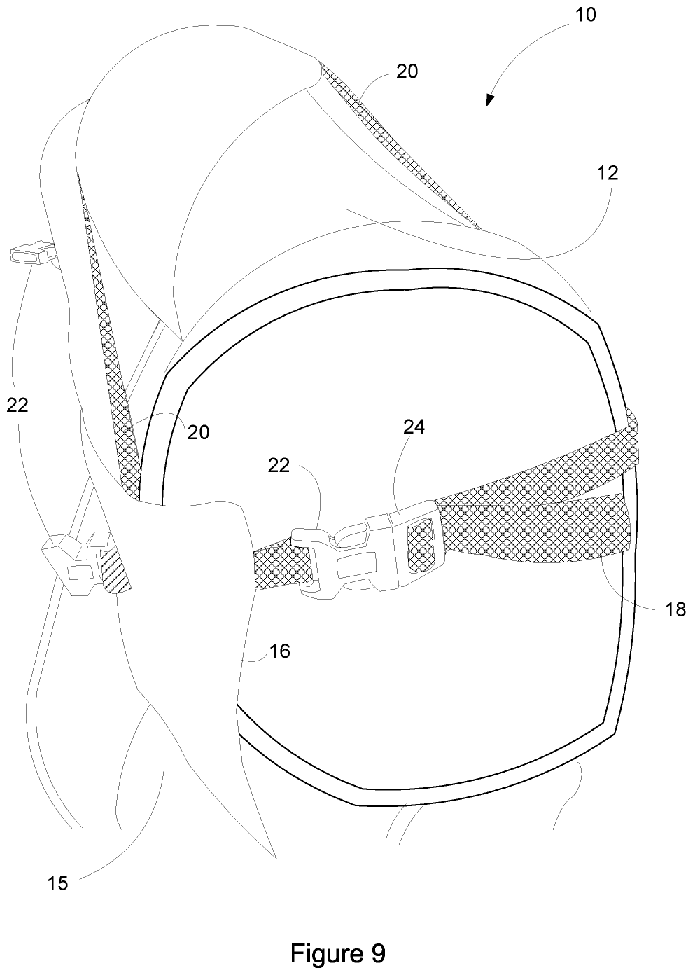

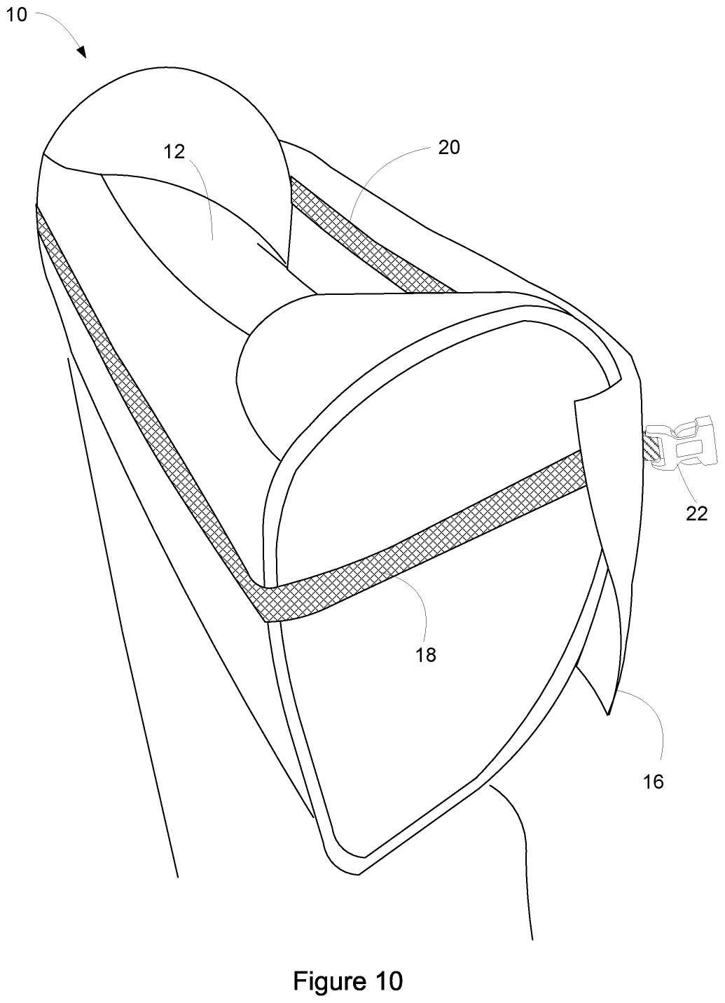

FIG. 8 is a front perspective view of the example hygienic headrest 12 accessory system of FIG. 6 operating on the seat 10 of FIG. 2. FIG. 9 is a front left perspective view of the example hygienic headrest 12 accessory system of FIG. 6 operating on the seat 10 of FIG. 2. FIG. 10 is a right rear perspective view of the example hygienic headrest 12 accessory system of FIG. 6 operating on the seat 10 of FIG. 2. Advantageously, the system can be attached to the headrest 12 of the traveller's seat 10 despite the minimal footprint that a traveller has in their seat 10. Further advantageously, the releasable buckles provide a manipulative solution for manipulating travel accessories, as will be illustrated further in the following figures. Yet further advantageously, the rectangular textile component 16 provides a hygienic covering to the headrest 12 for the traveller. Still yet further advantageously, the headrest strap 18 is dimensioned and can be positioned onto the headrest 12 so as to not interfere with an on-board entertainment system (not illustrated) of the seat 10 immediately behind the traveller, if present, by being advantageously at the top of the headrest 12, while the releasable buckles can be provided at a different height.

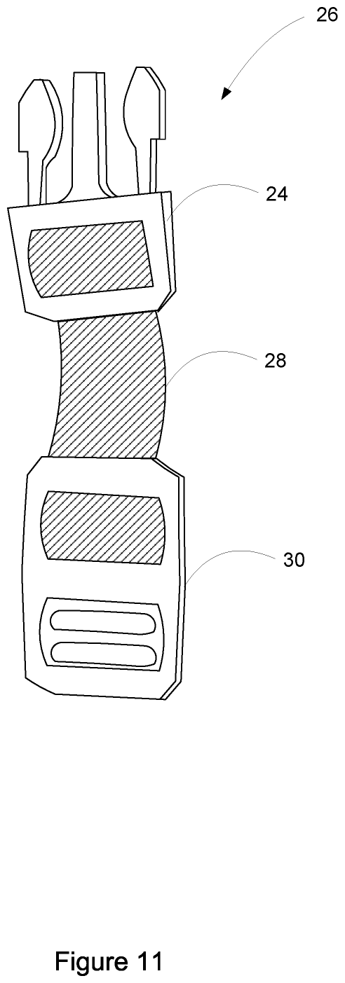

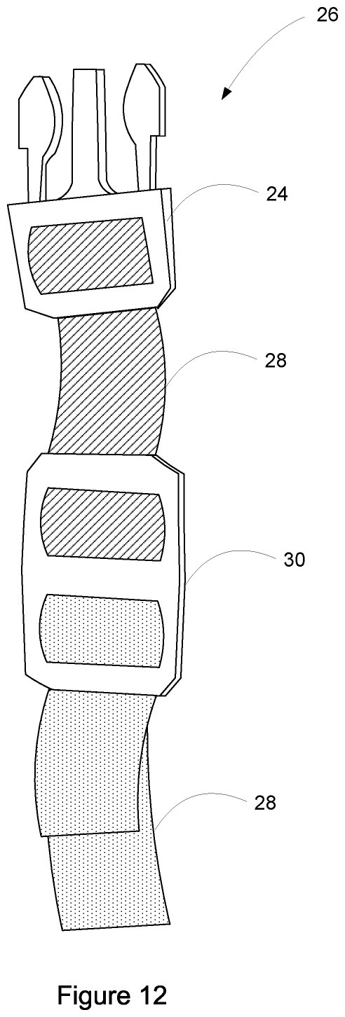

FIG. 11 is a front perspective view of an example accessory attachment 26 component. FIG. 12 is a front perspective view of an example accessory attachment 26 component with a strap 28 attached. The component includes a releasable buckle 22 that is compatible with the buckles described in relation to FIG. 8. To the buckle is attached a strap 28, not necessarily made of the same materials as the headrest strap 18 sewn onto the rectangular textile component 16, as well as a strap buckle 30 able to receive a strap 28, such as that of an eye mask, for example.

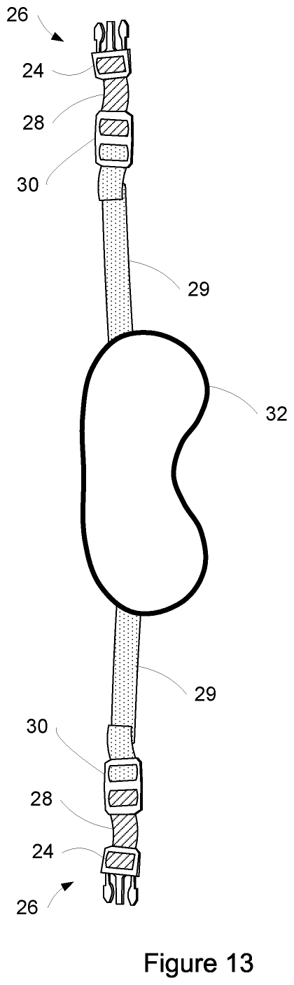

FIG. 13 is a front perspective view of an example eye mask accessory 32 component using two of the accessory attachment 26 components of FIG. 11. Advantageously, the eye mask strap 29 can be cinched at each side, thereby facilitating the manipulation of the eye mask when the eye mask is clipped into each of a releasable buckle 22 of the system.

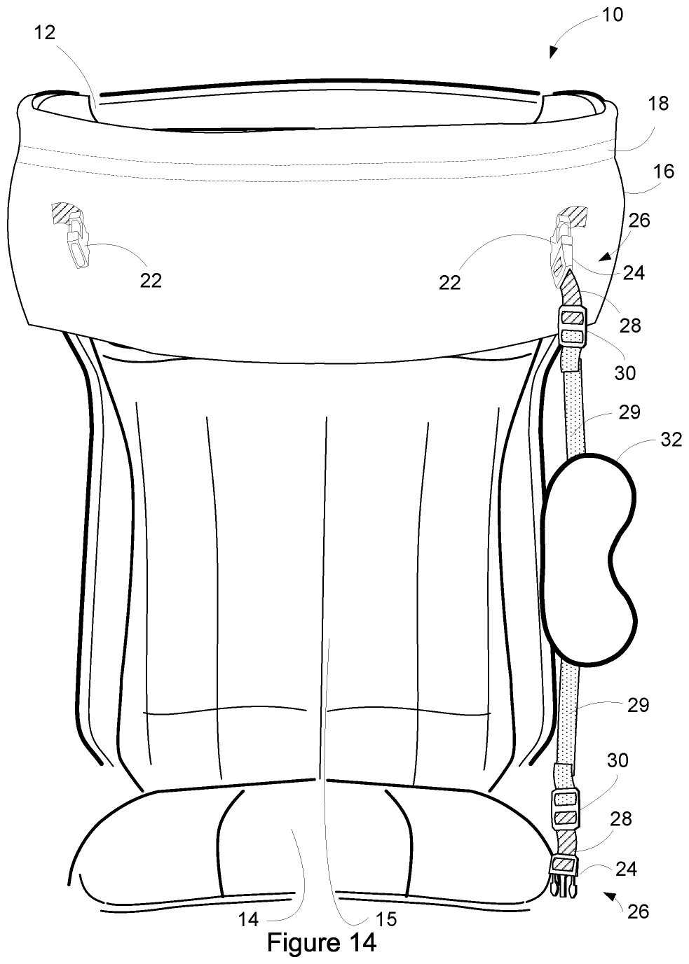

FIG. 14 is a front perspective view of the example hygienic headrest 12 accessory system of FIG. 6 cooperating with the seat 10 of FIG. 2 and the eye mask accessory 32 of FIG. 13. Advantageously, when not in use, the eye mask can be clipped into only one of the releasable buckle 22 provided on the headrest 12, thereby facilitating manipulation of the eye mask in a stowed position that is at a ready for use by simple manipulation of only one end of the eye mask strap 29, an advantage given the limited footprint provided to the traveller.

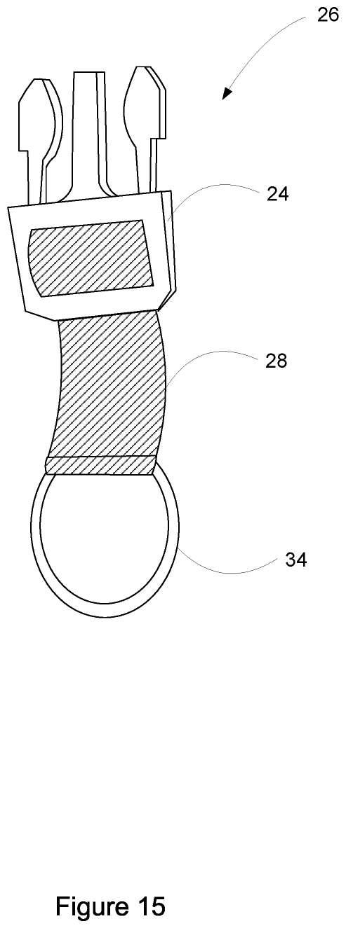

FIG. 15 is a front perspective view of an example accessory attachment 26 component. Instead of a strap buckle 30 of FIG. 11, a ring 34, such as a key ring 34, is provided, as a convenience to the traveller for manipulating accessories into a stowed and at ready position, again an advantage given the limited footprint provided to the traveller.

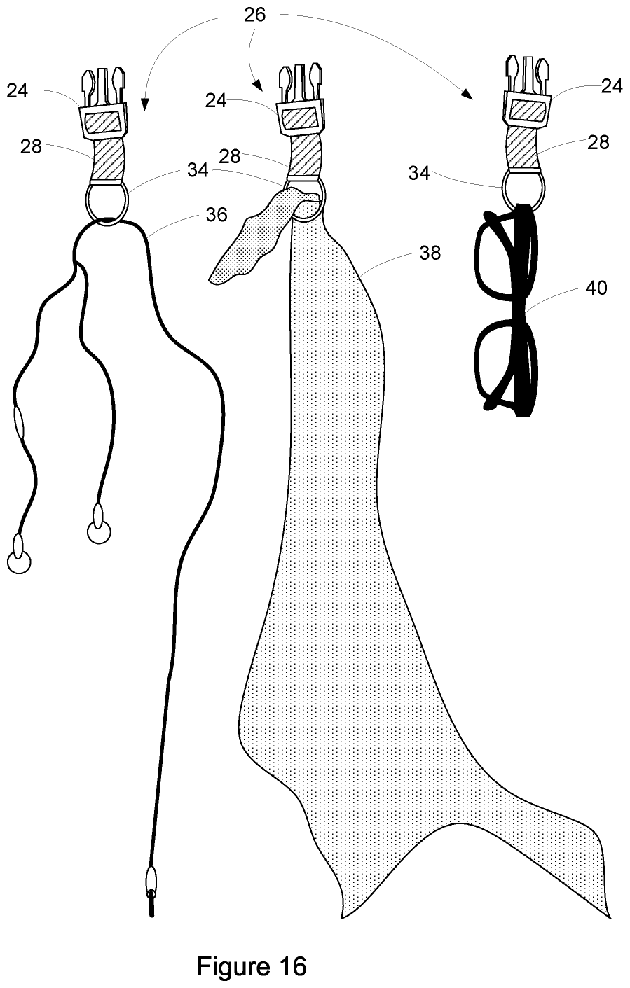

FIG. 16 is a front perspective view of three example accessory attachment 26 components of FIG. 15 for improved manipulation of three different example travel accessories. As shown, advantageously, the component of FIG. 15 can be used to stow at a ready a set of headphones 36, a blanket 38, or a pair of eyeglasses 40.

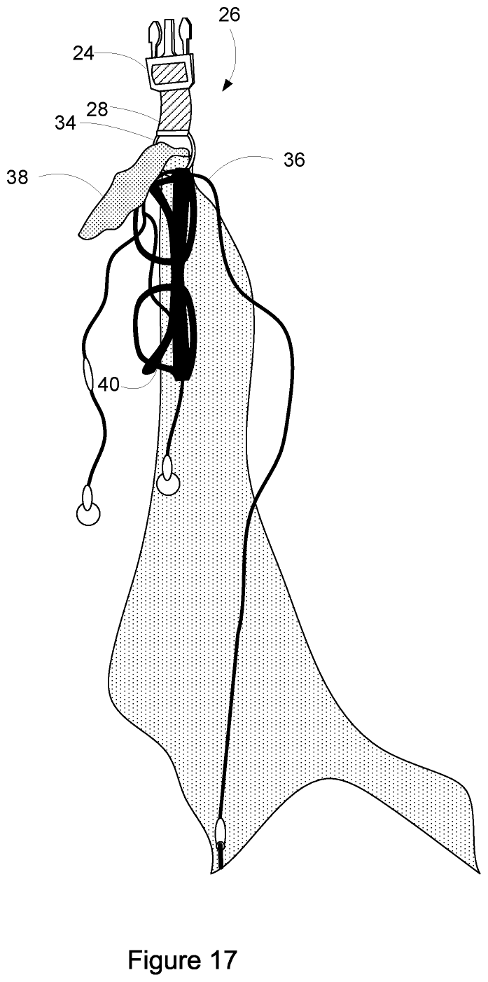

FIG. 17 is a front perspective view of one example multiple accessory attachment 26 component of FIG. 15 for improved manipulation of three different example travel accessories. As shown, advantageously, a single component of FIG. 15 can be used to stow at ready multiple accessories at once, such as a set of headphones 36, a blanket 38, and a pair of eyeglasses 40.

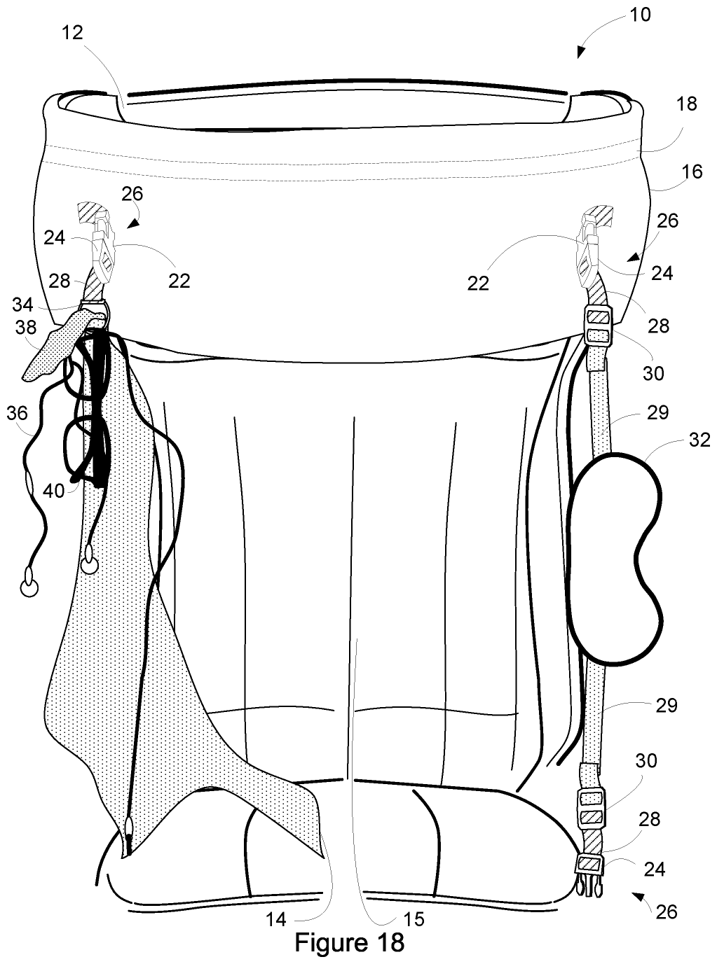

FIG. 18 is a front perspective view of the example hygienic headrest 12 accessory system of FIG. 6 operating on the seat 10 of FIG. 2, the eye mask accessory 32 of FIG. 13, and the multiple accessory attachment 26 component of FIG. 15. Advantageously, a traveller's accessories are stowed in an at-ready state despite the minimal footprint afforded to the traveller, while the headrest 12 is adapted for hygienic rest of the head of the traveller.

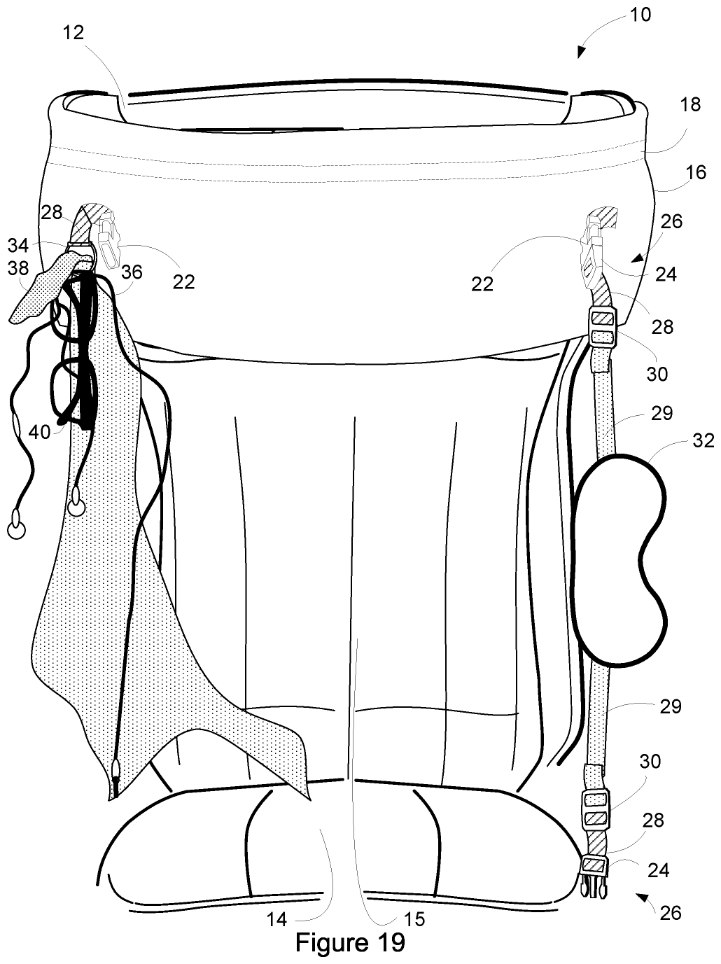

FIG. 19 is a front perspective view of an alternative example hygienic headrest 12 accessory system operating on the seat 10 of FIG. 2, the eye mask accessory 32 of FIG. 13, and integrating the multiple accessory attachment 26 feature of FIG. 15. Advantageously, the eye mask can be utilized while the accessories remain in a stowed and ready state. Advantageously, the accessory attachment 26 using a ring 34 is provided with a strap 28 sewn such that this feature is integrated into the rectangular textile component 16 on the manipulative side. As shown, the releasable buckle 22 component on the right side remains available to receive the corresponding releasable buckle 24 of the eye mask accessory 32 while simultaneously ensuring that the traveller's travel accessories remain in an at a ready state.

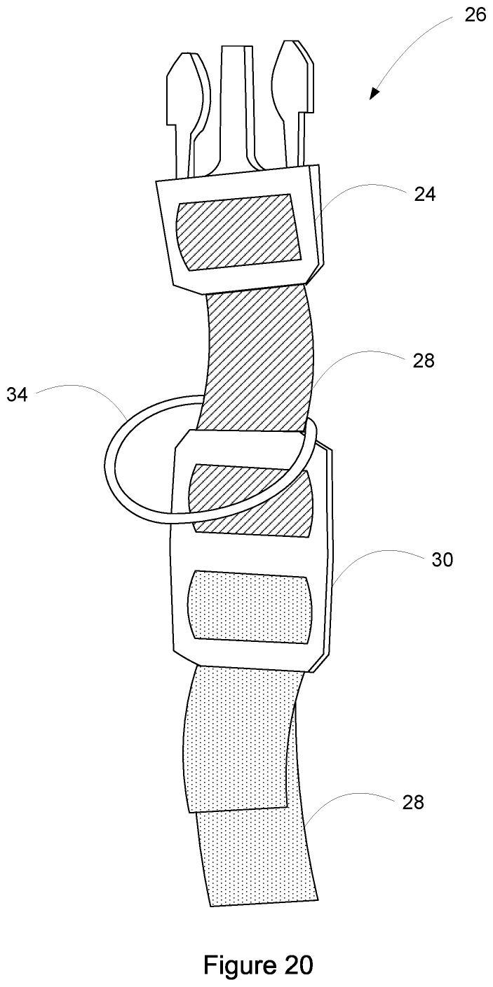

FIG. 20 is a front perspective view of an alternative example of FIG. 12 integrating the multiple accessory attachment 26 feature of FIG. 15. The ring 34 is provided between the corresponding releasable buckle 24 and the strap buckle 30 and is dimensioned to stay captive by the strap 28 there between. The ring 34 can be a key ring 34 or a rubber ring 34 like a rubber band.

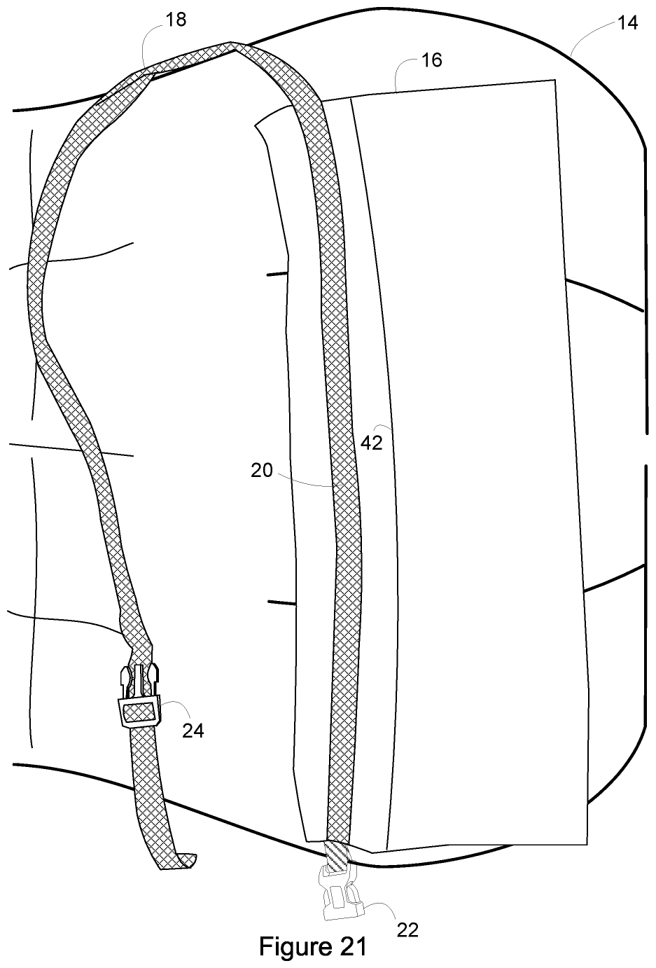

FIG. 21 is a top perspective view of the operational side of an alternative example hygienic headrest 12 accessory system. In addition to the headrest strap 18, non-slip component 20, releasable buckle 22, and corresponding releasable buckle 24, a pocket 42 is provided in the rectangular textile component 16 for stowing and retrieving components of the system, travel accessories, or other items as will be shown further below.

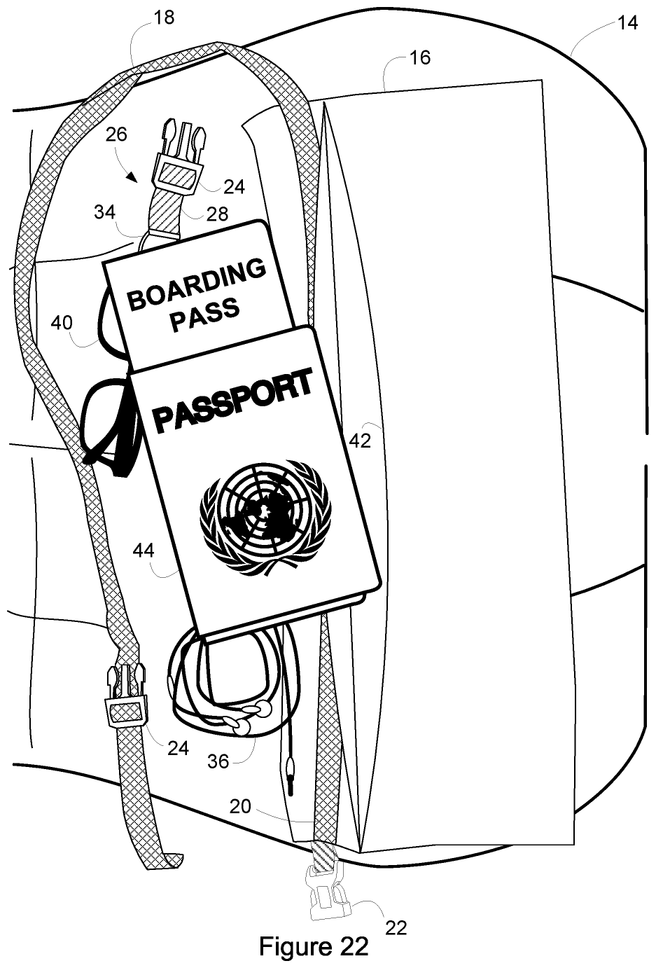

FIG. 22 is a top perspective view showing the pocket 42 feature of the alternative example hygienic headrest 12 accessory system of FIG. 21. The rectangular textile portion is doubled and a slit is provided to form a pocket 42 on the inside so as to store items and accessories that are not in use, such as unused accessory attachment 26 components, travel accessories, and travel documents 44 such as a boarding pass and passport, wallet, money, credit cards, cellular phone, keys, etc. Advantageously, items stowed in the pocket 42 are secure behind their head, whenever the traveller is seated, and especially when sleeping. Further advantageously, the pocket 42 allows the traveller to use the system as a money belt or fanny pack while boarding and leaving the plane, train, bus, car, etc. for both convenience, and security as the system can be hidden under layers of traveller's clothing, thereby reducing the risk of theft. Further advantageously, the pocket 42 can be stuffed with a pillow when provided in some flights, trains, or busses, or any other textile item such as a blanket 38, shirt, sweater, coat, etc. to create a pillow that can be fixed to the headrest 12 of the seat 10 for added comfort and security.

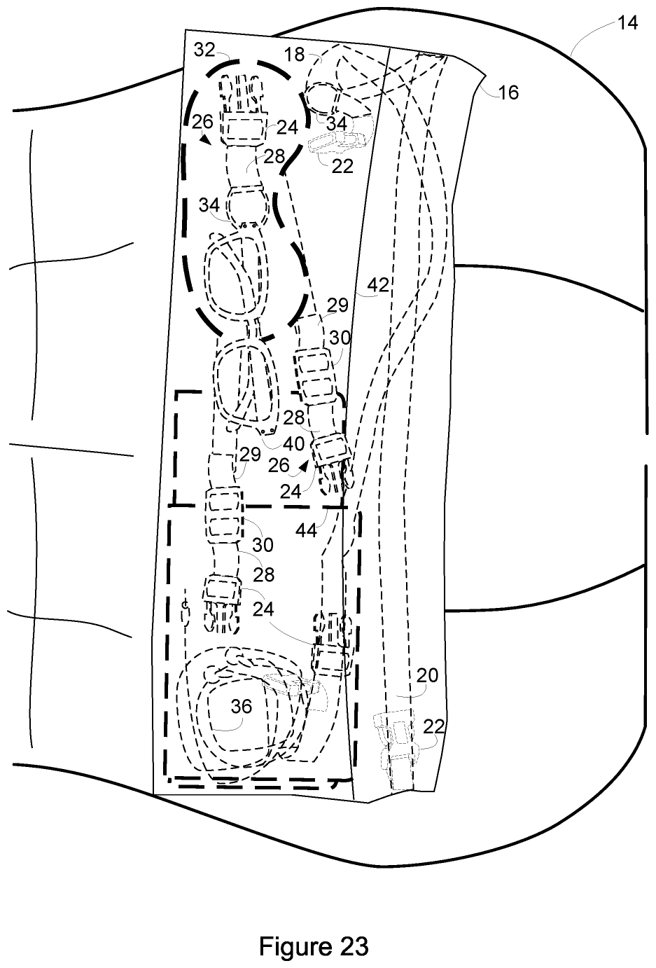

FIG. 23 is a top perspective view showing the reversible storage feature of the alternative example hygienic headrest 12 accessory system of FIG. 21. Advantageously, the pocket 42 can be fully inverted inside-out, thereby providing a way of storing all of the components of the system for shipping and handling during sales, and for compact manipulation by the traveller, who can also store travel accessories, documents, etc. in the system in this reversed configuration.

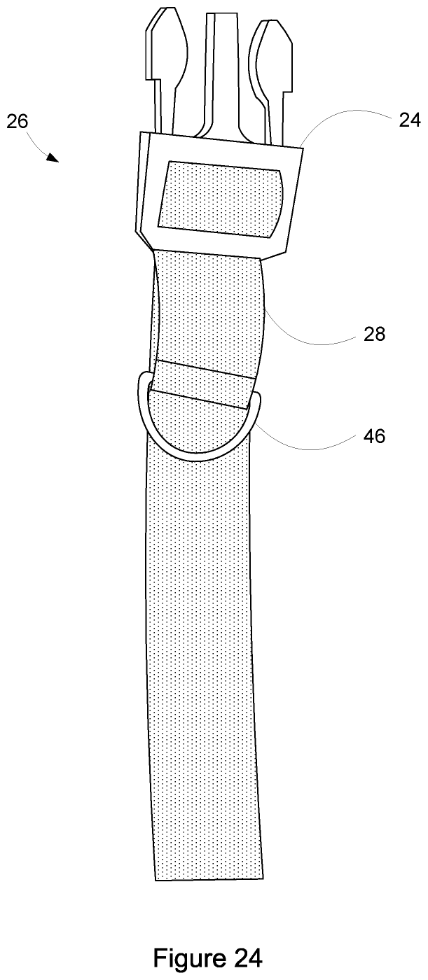

FIG. 24 is a front perspective view of an example accessory attachment 26 component having a D-ring 46. The D-ring 46 is particularly advantageous to enable one to cinch the strap 28 by manipulation in tight quarters, in the dark, or by feel alone when one's eyes are closed or covered with an eye mask and therefore ideal to provide in an eye mask strap 29 where corresponding releasable buckle 24 would be clipped into releasable buckle 22 of the rectangular textile are proximate to one's temple. Furthermore, the D-ring 46 can double as a ring 34 for holding other travel accessories as has been illustrated in other drawings using a circular ring 34.

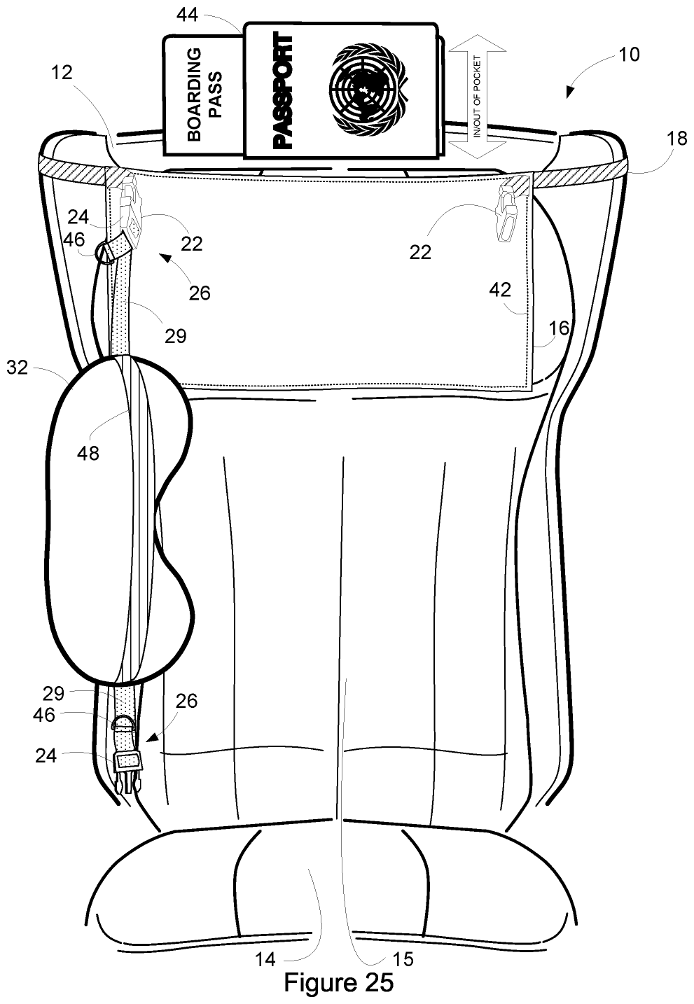

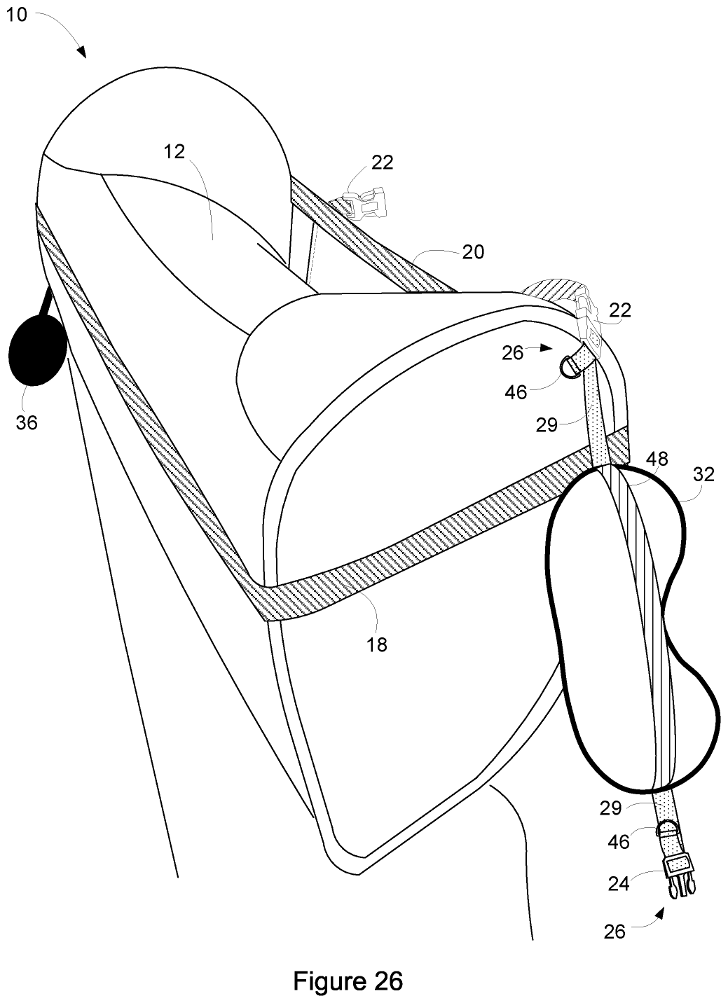

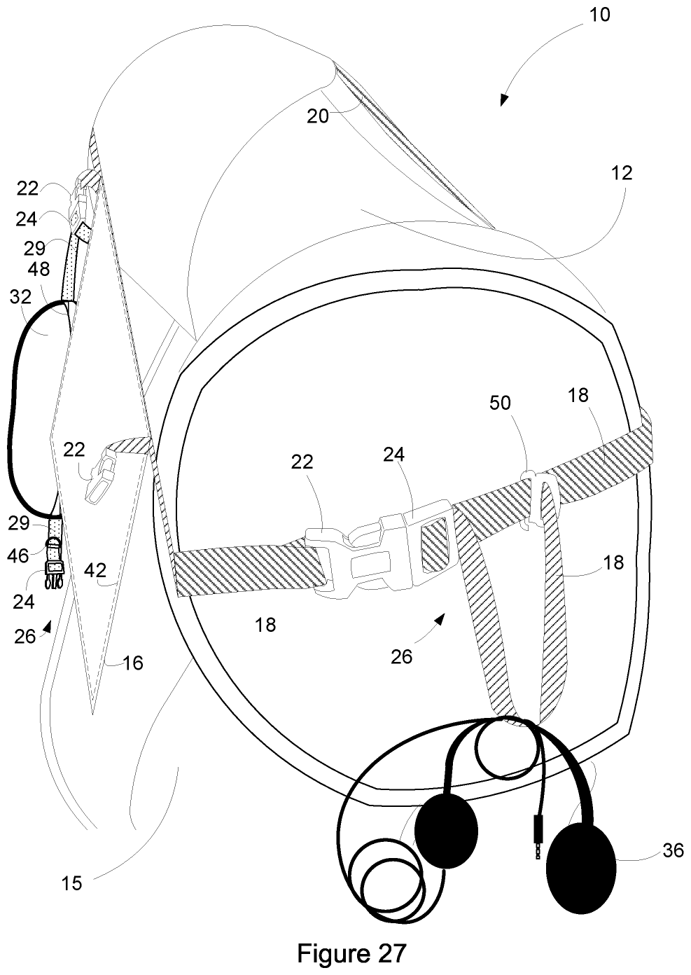

FIG. 25 is a front perspective view of an example eye mask accessory 32 component using two of the accessory attachment 26 components of FIG. 24 to attach to an example hygienic headrest 12 accessory system operating on the seat 10 of FIG. 2. FIG. 26 is a right rear perspective view of the example hygienic headrest 12 accessory system of FIG. 25 operating on the seat 10 of FIG. 2. FIG. 27 is a front left perspective view of the example hygienic headrest 12 accessory system of FIG. 25 operating on the seat 10 of FIG. 2. In addition to the use of the accessory attachment 26 component of FIG. 24, an elastic strap 48 is provided on the eye mask accessory 32 to facilitate securing the eye mask over one's eyes. As compared with the rectangular textile component 16 illustrated in other Figures, the one of FIG. 25 is more compact, provides the headrest strap 18 in two parts, each at a respective end of the rectangular textile component 16, and has a pocket 42 for putting in and taking out items such as the travel documents 44 illustrated. Advantageously, a clip 50 is provided on the loose end of the headrest strap 18 so that the slack can be taken up to keep the headrest strap 18 taught, or as illustrated to create a loop to hold travel accessories such as the headphones 36 illustrated.

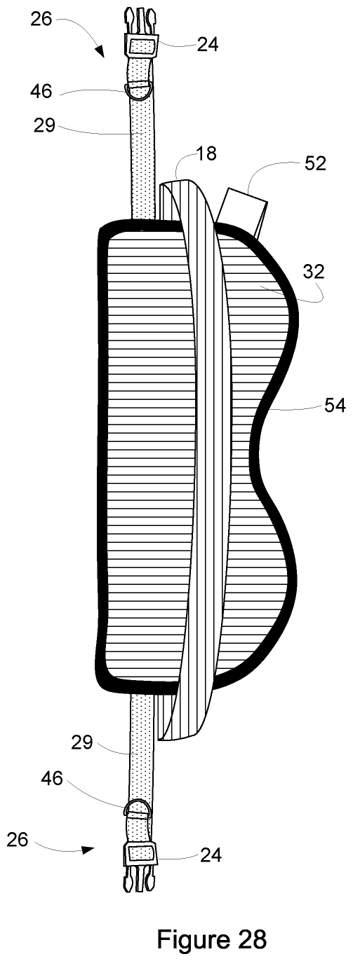

FIG. 28 is a front perspective view of the example eye mask accessory 32 component of FIG. 25. Advantageously, the eye mask strap 29 can be cinched at each side using the provided corresponding D-ring 46, thereby further facilitating the manipulation of the eye mask when the eye mask is clipped into each of a releasable buckle 22 of the system. Further advantageously, an elastic strap 48 is provided on the eye mask accessory 32 so as to facilitate placement onto a traveller's head and to keep the eye mask covering their eyes whether the eye mask accessory 32 is clipped onto the rectangular textile area or not. Further advantageously, a piping 54 is provided on the periphery of the eye mask accessory 32 to provide a tighter seal with the varied faces of travellers and for improved comfort. Yet further advantageously, a label 52 is provided for branding purposes such that a service provider could brand the system to provide to travellers for promotional purposes, or to advise a traveller that they are using the original system and not a counterfeit of the original.

FIG. 14 is a front perspective view of the example hygienic headrest 12 accessory system of FIG. 6 cooperating with the seat 10 of FIG. 2 and the eye mask accessory 32 of FIG. 13. Advantageously, when not in use, the eye mask can be clipped into only one of the releasable buckle 22 provided on the headrest 12, thereby facilitating manipulation of the eye mask in a stowed position that is at a ready for use by simple manipulation of only one end of the eye mask strap 29, an advantage given the limited footprint provided to the traveller.

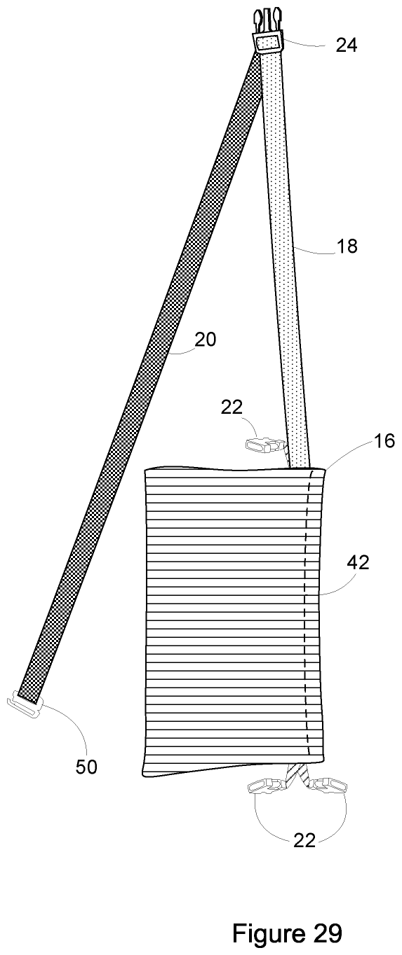

FIG. 29 is a front perspective view of an alternative to the example hygienic headrest 12 accessory system of FIG. 25. As can be seen, instead of only one releasable buckle 22 being provided at each end of the rectangular textile component 16, on one end a pair of releasable buckle 22 are provided thereby ensuring that at least one other travel accessory can be clipped onto the system while an eye mask accessory 32 is clipped onto the system.

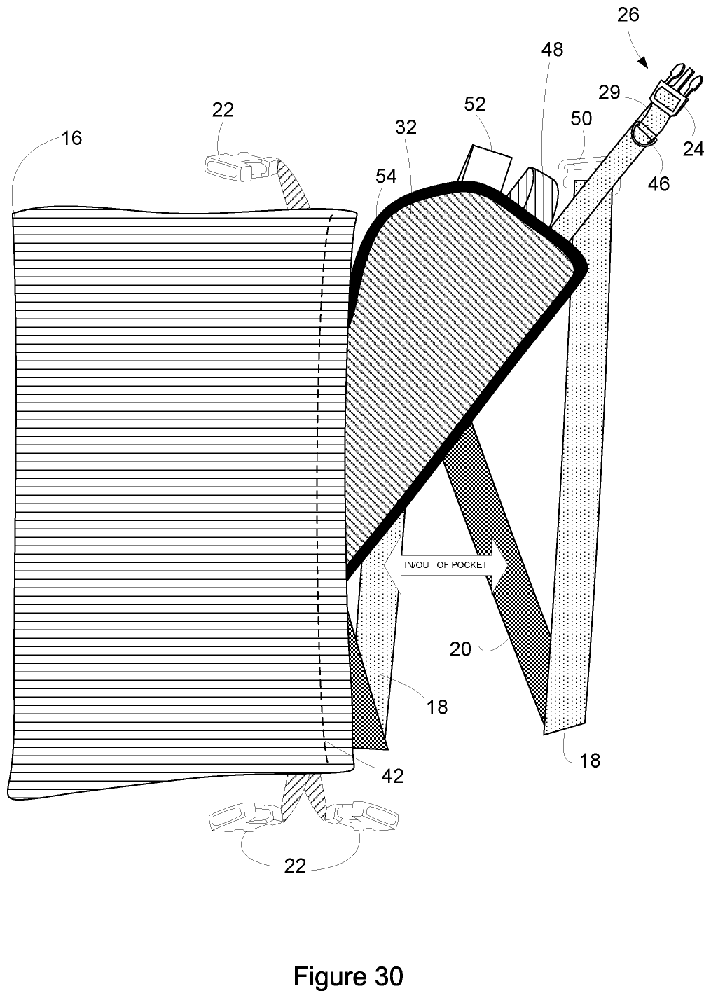

FIG. 30 is a top perspective view of a pocket 42 feature of the example hygienic headrest 12 accessory system of FIG. 29. As illustrated, components of the system and other items can be stowed in and out of the pocket 42 provided in the rectangular textile area.

The above-described embodiments of the present disclosure are intended to be examples only. Those of skill in the art may effect alterations, modifications and variations to the particular embodiments without departing from the scope of the disclosure.

What is claimed is:

1. A hygienic travel headrest accessory system comprising:
an elongate textile component having an operative side for covering a portion of a headrest of the seat of a traveller and a manipulative side for abutting against the head of the traveller;
a headrest strap connected to the textile component at one end and extending freely for a predetermined distance thereafter sufficient to go around the headrest, the headrest strap having an operative side with a non-slip component;
a cinching mechanism connecting the free end of the headrest strap to the other end of the textile component for cinching the headrest strap and textile component around the headrest thereby positioning the textile component between the headrest and the head of the traveller; and
an accessory attachment component provided at least on one end of the textile component for receiving travel accessories;
wherein the traveller can use the cinching mechanism to both configure the headrest strap to cinch around the headrest so as to ensure that the headrest strap remains in place when cinched around the headrest while positioning the textile component hygienically between the headrest and the travellers head and to secure the accessory attachment component for receiving travel accessories in a stowed and at ready position or in an in use position.

2. The system of claim 1, wherein the cinching mechanism includes a releasable buckle component provided at the other end of the textile component that is compatible with a corresponding releasable buckle provided on the free end of the headrest strap.

3. The system of claim 1, further comprising a clip provided on the free end of the headrest strap such that the free end can be clipped onto the headrest strap to form a loop for receiving accessories.

4. The system of claim 1, wherein the accessory attachment component includes a releasable buckle component.

5. The system of claim 4, further comprising an eye mask accessory, the eye mask accessory comprising:
an elongate eye covering component configured to cover the eyes of the traveller; and
a pair of eye mask straps, each eye mask strap having two ends, the first end fixed to a respective end of the eye covering component, and the second end having a corresponding releasable buckle that is compatible with the releasable buckle component of the accessory attachment component of the textile component;
whereby the eye mask can be releasably clipped into at least one end of the textile component when in the stowed and at ready position, and to both ends of the textile component when in the in use position.

6. The system of claim 5, wherein the eye mask accessory further includes an elastic strap for securing the eye mask to the travellers head.

7. The system of claim 5, wherein the eye mask accessory further includes a D-ring provided at the end of each of the eye mask strap to facilitate manipulation for cinching or loosening the eye mask strap.

8. The system of claim 5, wherein the eye mask accessory further includes a piping provided on the periphery of the eye covering component to provide a tighter seal or improved comfort.

9. The system of claim 5, wherein the eye mask accessory further includes a label for branding purposes.

10. The system of claim 1, further comprising a pocket feature provided in the textile component for securely stowing items while the traveller is resting their head on the textile component, for stowing components of the system, or for packaging the components of the system.

11. The system of claim 10, wherein the pocket feature is used to store travel documents.

12. The system of claim 1, wherein the accessory attachment component is configured to receive an eye mask.

13. The system of claim 1, wherein the accessory attachment component is configured to receive a pair of glasses.

14. The system of claim 1, wherein the accessory attachment component is configured to receive a blanket.

15. The system of claim 1, wherein the accessory attachment component is configured to receive headphones.
